(19) United States
(12) Patent Application Publication
(10) Pub. No.: US 2019/0247183 A1
(43) Pub. Date: Aug. 15, 2019

(54) PROSTHESIS FOR NOSE SURGERY

(71) Applicant: Tae Hwan AHN, Seoul (KR)

(72) Inventor: Tae Hwan AHN, Seoul (KR)

(21) Appl. No.: 16/336,882

(22) PCT Filed: Aug. 30, 2017
(86) PCT No.: PCT/KR2017/009493

§ 371 Date: Mar. 26, 2019

(30) Foreign Application Priority Data
Sep. 27, 2016 (KR) 10-2016-0124026

(51) Int. Cl. A61F 2/18; A61L 27/10; A61L 27/04

(52) U.S. Cl. CPC A61F 2/186; A61L 27/58; A61L 27/04; A61L 27/10

(57) ABSTRACT

A prosthesis for nose surgery is disclosed. The present invention comprises: a column part; a first fixing part having a first leg part and a second leg part which are divided into two parts at one side end of the column part and extend therefrom; and a second fixing part provided at the other side end of the column part. The prosthesis for nose surgery according to the present invention improves the structural integrity of a skeleton forming a nasal valve by reinforcing a columella when the nasal valve is constricted due to inherent or acquired anatomical anomalies such as nasal septum deviation, thereby resolving nasal valve stenosis and as a result, smoothening a nasal air flow.

### CROSS-REFERENCE TO RELATED APPLICATIONS

A claim for priority under 35 U.S.C. § 119 is made to Korean Patent Application No. 10-2016-0124026 filed on Sep. 27, 2016 in the Korean Intellectual Property Office, the entire contents of which are hereby incorporated by reference.

### TECHNICAL FIELD

The present invention relates to a prosthesis for nose surgery, and more particularly, to a prosthesis for nose surgery disposed in a columella (a nose pillar) when inserted into a nose thereby improving structural integrity of a skeleton configuring a nasal valve.

### BACKGROUND ART

As people have shown interest in cosmetics, interest in plastic surgery has also increased recently. In particular, since a nose occupies a center of a face, the nose plays a central role in formation of a facial contour and overall impression. Asians have lower noses and wider nostrils than Westerners and tend to look wider overall so that the number of men and women of all ages who want nose surgery has increased.

In addition to aesthetic purposes, with respect to a case to a damaged nose due to injury or a case to an unnatural-shape nose which is inherent or acquired, augmentation rhinoplasty which makes a nasal dorsum higher, corrective rhinoplasty which makes a crooked nasal tip corrective, and graft rhinoplasty of nasal septal extension plate which reinforces a columella to improve structural integrity of a skeleton configuring a nasal valve.

In particular, the nasal valve, which is a narrow organ in the nose, allows airflow to flow in and out of the nose and plays a pivotal role in controlling respiration. When the nasal valve is constricted due to anatomical anomalies which are inherent or acquired, such as septal deviation, a nasal obstruction symptom occurs. To solve this symptom, it is necessary to improve structural integrity of the skeleton configuring the nasal valve. As one method of such improvement, nose surgery for expanding a nasal septum, in which a prosthesis is drafted to reinforce the columella, has been used.

In augmentation rhinoplasty making a nasal dorsum higher, medical silicone or Gore-Tex is generally used as an artificial material, which is inserted into the nose along a longitudinal direction of the nose. In addition, polyethylene terephthalate, Proplast I and II (Medpore™), or the like may be used. Since these materials are rigid and are inserted outside the nasal dorsum, it is suitable to make the entire nose higher. However, it is difficult to be applied when these materials reinforce the columella to alleviate the nasal valve stenosis symptom because protrusion and inflammatory response occur in the nose. Due to this problem, when the columella is reinforced to expand the nasal septum, a method using a relatively rigid autologous cartilage is used. In conventional surgery in reinforcing the columella, a part of a nasal septal cartilage, which is taken from a patient and is processed for a specific purpose, or a costal cartilage, which is taken from the patient and is processed, has been used as a pillar for reinforcing the columella. However, when the autologous cartilage, such as the nasal septal cartilage or the costal cartilage, is used, the cartilage is deformed by infection of bacteria and is melted to be absorbed, thereby causing a shape deformation of the nose.

In addition, since the columella is artificially raised through autologous cartilage grafting, the autologous cartilage grafted in the columella receives force, which intends to return to an original low nose state, that is, a downward pressure after surgery. The autologous cartilage is not rigid enough to withstand the downward pressure, so that the cartilage is easily bent or damaged after surgery. Also, after a long time since surgery, the cartilage deviates from an original surgical position.

Furthermore, since noses of Asians are flatter than Westerns and a nasal septal cartilage configuring a nasal septum is smaller and less rigid compared to Westerners, the amount of the nasal septal cartilage from the nose for autologous cartilage grafting is limited. In the case of the costal cartilage, an additional operation is required to incise a costa in addition to the nose, and scars of the costa occur.

### DISCLOSURE OF INVENTION

### Technical Problem

The present invention has been made to solve the above problems, and it is an object of the present invention to provide a prosthesis, instead of an autologous cartilage of a patient, for nose surgery which reinforces a columella for function improvement of a nasal valve, thereby preventing a patient's cartilage or other body parts to be damaged and performing a simplified procedure of nose surgery.

In addition, the present invention also provides a prosthesis which is secure from bacterial infection and is sufficiently rigid instead of an autologous cartilage, which is vulnerable to bacterial infection and not sufficiently rigid, thereby keeping a shape and function of a patient' nose after nose surgery.

### Solution to Problem

In order to achieve the above object, a prosthesis for nose surgery according to the present invention including a column part 110; a first fixing part 120 including a first leg and a second leg which are divided from one end of the column part 110 to be extended; and a second fixing part 130 disposed at the other end of the column part 110.

Desirably, the column part 110 may be disposed in a columella when inserted into the nose.

Furthermore, the first fixing part 120 may be fixed to a posterolateral part 210 of a nasal septal cartilage 200.

Furthermore, the second fixing part 130 may be fixed between both alar cartilages 300.

Furthermore, the second fixing part 130 may further include a fixing groove 125 formed between the first and second legs 122 and 124 and the posterolateral part 210 of the nasal septal cartilage 200 may be coupled to the fixing groove 125.

Furthermore, the first leg 122 may have a first through hole 126 passing through the first leg 122, the second leg 124 may have a second through hole 128 passing through the second leg 124, and a central axis of the first through hole 126 and a central axis of the second through hole 128 may be collinear.

Furthermore, the second fixing part 130 may have a third through hole 132 passing through the second fixing part 130.

Furthermore, a plurality of the third through holes 132 is formed at the column part 110 in a longitudinal direction of the second fixing part 130.

Furthermore, the column part 110 may include an outer shell portion 112 formed of a first material and a core portion 114 formed of a second material, and the outer shell portion 112 surrounds a surface of the core portion 114.

Furthermore, the second material may include a biomedical metal.

Furthermore, the first fixing part 120 may be fixed to the posterolateral part 210 of the nasal septal cartilage 200 by a suture passing through the first through hole 126, the posterolateral part 210 of the nasal septal cartilage 200, and the second through hole 128.

Furthermore, the suture may be decomposed and disappear in a body when a predetermined period of time elapses, and the first and second through holes 126 and 128 may be filled with a living tissue to be integrally adhered to a surround tissue.

Furthermore, the second fixing part 130 may be fixed between the both alar cartilages by a suture passing through one alar cartilage, at least one of the third through holes, and the other alar cartilage.

Furthermore, the suture may be decomposed and disappear when a predetermined period of time elapses, and the third through holes 132 may be filled with a living tissue to be integrally adhered to a surround tissue.

Furthermore, the prosthesis may be formed of a ceramic material.

Furthermore, the first material may be formed of a ceramic material.

Furthermore, the first material may be formed of a zirconia material.

Furthermore, the column part may have a cylinder shape.

Furthermore, the column part may have a diameter of 2 mm to 4 mm.

Furthermore, the prosthesis may have a length of 1 cm to 5 cm.

### Advantageous Effects of Invention

As described above, the prosthesis for nose surgery improves structural integrity of the skeleton configuring the nasal valve by reinforcing the columella when the nasal valve is constricted due to anatomical anomalies which are inherent or acquired, such as septal deviation, thereby relieving stenosis of the nasal valve and, consequently, facilitating flow of the nasal airflow.

In addition, in the case of using the prosthesis for nose surgery according to the present invention, since it is not necessary to take the nasal septal cartilage or the costal cartilage from the patient for reinforcing the columella, the autologous cartilage is reserved, and therefore malfunction or defunctionalization of the autologous cartilage is prevented.

Also, in the case of using the prosthesis for nose surgery according to the present invention, since it is not necessary to take the costal cartilage which is an autologous cartilage, the procedure of nose surgery may be simplified. In addition, since it is not necessary to incise the skin around the costa besides the nose, nose surgery may be performed without the scars around the patient's costa.

Furthermore, the prosthesis for nose surgery according to the present invention is formed of a ceramic material or a biomedical metal, which does not generate absorption or deformation when infection of bacteria and has excellent stiffness compared to the nasal septal cartilage or the costal cartilage. Therefore, in the case of exposure of postoperative infection risk or external impact to the surgical site, the shape of patient's nose may remain permanently and function of the nasal valve may not be deteriorated after a long period of time.

In addition, in the prosthesis for nose surgery according to the present invention, with the first fixing part and the second fixing part are disposed at both ends of the column part, respectively, the both ends of the column part may be adhered to tissues corresponding to the both ends of the column part, and therefore the prosthesis may be firmly fixed within the nose.

Also, the prosthesis for nose surgery according to the present invention is disposed in the columella along a longitudinal direction of the nose, the first fixing part is fixed at the posterolateral part of the nasal septal cartilage, and the second fixing part is coupled between the both alar cartilages. Therefore, the prosthesis reinforces the columella at the optimal location within the nose to improve structural integrity of the skeleton configuring the nasal valve.

Furthermore, in the prosthesis for nose surgery according to the present invention, the posterolateral part of the nasal septal cartilage is disposed to be coupled to the fixing groove disposed between the first and second legs of the first fixing part. The first fixing part of the prosthesis is firmly fixed to the posterolateral part, which is located near anterior nasal spine of tibia and is relatively thick and rigid in the nasal septal cartilage.

Also, in the prosthesis for nose surgery according to the present invention, since the first through hole is formed in the first leg and the second through hole is formed in the second leg, the surgical needle and the suture may freely pass through the first fixing part including the first and second legs which is made of ceramic having a high hardness.

In addition, in the prosthesis for nose surgery according to the present invention, since a plurality of the third through holes is formed in the column part along a longitudinal direction of the column part, the most suitable third through hole among the plurality of third through holes may be selected based on the length of the columella and the position of the alar cartilage and the suture may pass through the selected third through hole. Thereby, the second fixing part may be properly coupled between the alar cartilages.

Furthermore, in the prosthesis for nose surgery according to the present invention, the first fixing part is sutured by the suture passing through the first through hole, the posterolateral part of the nasal septal cartilage, and the second through hole, so that the first fixing part and the posterolateral part of the nasal septal cartilage are overlapped and sutured in a three-layered way, thereby being firmly coupled to one another.

In addition, in the prosthesis for nose surgery according to the present invention, the second fixing part is sutured by the suture passing through one alar cartilage, at least one of the third through holes, and the other alar cartilage, so that the second fixing part and the both alar cartilages are overlapped and sutured in a three-layered way, thereby being firmly coupled to one another.

Also, in the prosthesis for nose surgery according to the present invention, the suture formed of the biodegradable polymer material is decomposed and disappears and the first to third through holes are filled with the living tissues to be integrally adhered to the surround tissues. Thereby, the first fixing part may be firmly fixed to the posterolateral part of the nasal septal cartilage and the second fixing part may be firmly fixed between the both alar cartilages.

### BRIEF DESCRIPTION OF THE DRAWINGS

FIG. 1 is a perspective view of a prosthesis according to the present invention,

FIG. 2A is a plan view of a prosthesis according to a first embodiment of the present invention,

FIG. 2B is a side view of the prosthesis according to the first embodiment of the present invention,

FIG. 3 is a schematic bottom view of a prosthesis according to the present invention, which is inserted into a nose,

FIG. 4 is a schematic side view of a prosthesis according to the present invention, which is inserted into a nose,

FIG. 5 is a plan view of the prosthesis according to a second embodiment of the present invention, and

FIGS. 6 to 9 are views illustrating inner and outer structures of a nose.

### DESCRIPTION OF EMBODIMENTS

Hereinafter, the present invention will be described in detail with reference to the drawings. It is to be noted that the same elements among the drawings are denoted by the same reference numerals whenever possible. In the following description, well-known functions or constructions are not described in detail since they would obscure the invention in unnecessary detail.

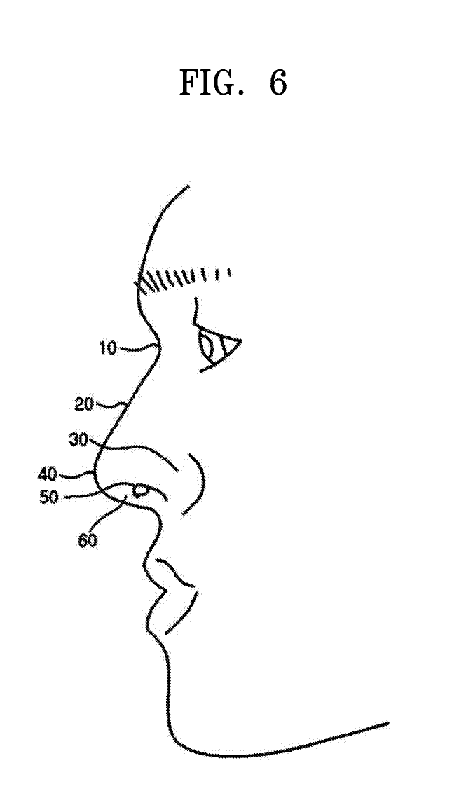

As shown in FIG. 6, an exterior of a human nose includes a dorsum 20 extending from a root 10 to an apex 40, curved alae 30 located on both sides of the apex 40, two nostrils 50, and a columella 60, which is located between the nostrils 50 and functions as a pillar supporting the apex 40.

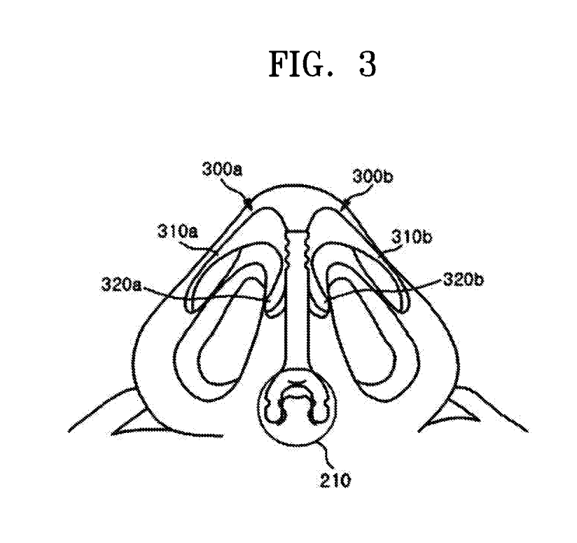

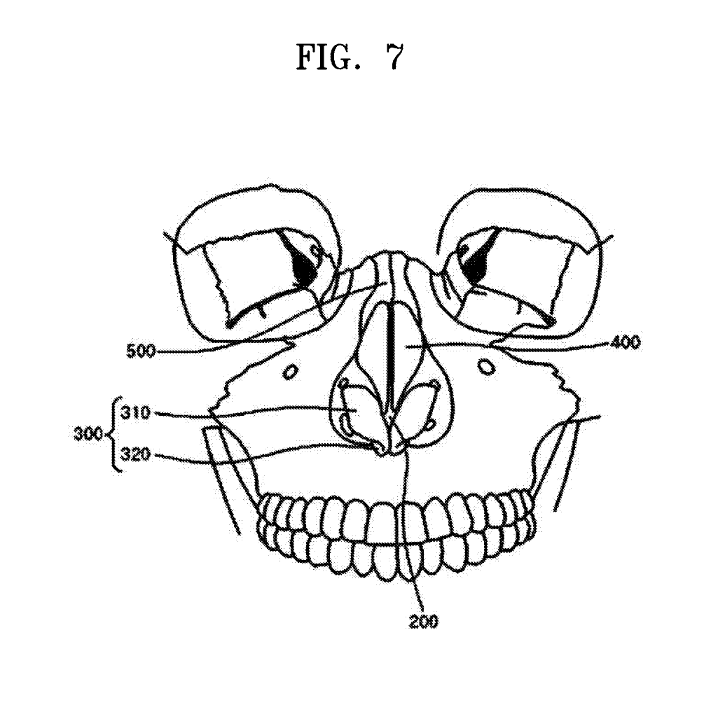

Furthermore, as shown FIGS. 3, 7 and 8, an interior of the human nose includes a nasal bone 500, a nasal septal cartilage 200, upper lateral cartilages 400, and lower lateral cartilages 300.

The nasal bone 500 occupies 1/3 of an upper nose and has a thin thickness. The nasal septal cartilage 200 is disposed at a center of the nose and divides the inside nose into left and right. Furthermore, the nasal septal cartilage 200 is elastic and rigid, supporting the dorsum 20 and the apex 40 at the inside dorsum 20 and apex 40. The nasal septal cartilage 200 morphologically serves as a center rod to determine length and height of the nose. The upper lateral cartilages 400 occupy ⅓ of a central nose and serve as a connection between the nasal bone 500 and the alae 30. The lower lateral cartilages 300, which are referred to as alar cartilages 300, 300a, 300b, includes outer cartilages 310, 310a, 310b and inner cartilages 320, 320a, 320b and serves to determine a shape of the apex 40.

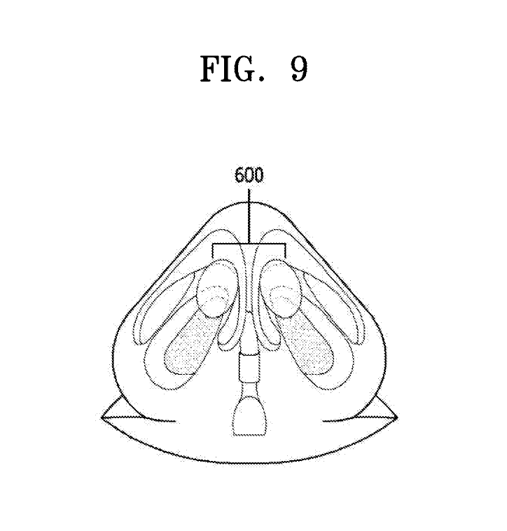

As shown in FIG. 9, a nasal valve 600 corresponds to a ceiling space at an upper end of the nose, and serves as a passage for airflow in the nose. When the nasal valve 600 is constricted due to anatomical anomalies which are inherent or acquired, such as septal deviation, a nasal obstruction symptom may occur. To solve these problems, it is necessary to improve structural integrity of a skeleton configuring the nasal valve 600. As one method of such improvement, nose surgery for expanding a nasal septum, in which a prosthesis is drafted to reinforce the columella 60, has been used.

In augmentation rhinoplasty making the dorsum 20 higher, medical silicone or Gore-Tex is generally used as an artificial material, which is inserted into the nose along a longitudinal direction of the nose. In addition, polyethylene terephthalate, Proplast I and II (Medpore™), or the like may be used. Since these materials are rigid and are inserted outside the dorsum 20, it is suitable to make the entire nose higher. However, it is difficult to be applied when these materials is inserted into the columella 60 to vertically support the apex 40 in order to alleviate the nasal valve stenosis symptom because protrusion and inflammatory response occur in the nose. Due to this problem, when the columella 60 is reinforced to expand the nasal septum, a method using a relatively rigid autologous cartilage is used.

In surgery for reinforcing the columella 60, a part of the nasal septal cartilage 200, which is taken from a patient and is processed for a specific purpose, or a costal cartilage, which is taken from the patient and is processed, has been used as a pillar for reinforcing the columella 60.

However, when the autologous cartilage, such as the nasal septal cartilage 200 or the costal cartilage, is used, the cartilage is deformed by infection of bacteria and is melted to be absorbed, thereby causing a shape deformation of the nose.

Furthermore, since the columella 60 is artificially extended through autologous cartilage grafting to make the apex 40 higher, and the autologous cartilage grafted in the columella 60 receives force, which intends to return to an original low nose state, that is, a downward pressure after surgery. The autologous cartilage is not rigid enough to withstand the downward pressure, so that the autologous cartilage is easily bent or damaged after surgery. Also, after a long time since surgery, the cartilage deviates from an original surgical position.

In addition, when the nasal septal cartilage 200 is transplanted, since the Asian nose is flatter than Westerns and the nasal septal cartilage 200 configuring the nasal septum is smaller and less rigid compared to Westerners, the amount of the nasal septal cartilage 200 from the Asian nose for grafting is limited. Furthermore, the nasal septal cartilage 200 has a non-uniform thickness. When a thin part of the nasal septal cartilage 200 is taken for grafting, a force for supporting the apex 40 is weakened. When a thick part of the nasal septal cartilage 200 is taken for grafting, a supporting force of the remaining nasal septum is weakened so that the remaining nasal septum may not support the dorsum 20 and the dorsum 20 may be collapsed.

Also, in the case of transplantation of the costal cartilage, a separate operation for collecting costal cartilage is required, an additional scar and pain in the patient's body occur, and overall operation time and effort are further required. In fact, a large number of patients with nose surgery are women, and these women tend to be reluctant to have scars around their costa, making it difficult to perform the operation to collect costal cartilage.

The present invention has been made to solve the above problems and it is an object of the present invention to provide a prosthesis instead of the autologous cartilage of the patient in nose surgery for reinforcing the columella 60 in order to improve function of the nasal valve 600. Therefore, a procedure of nose surgery may be performed without damaging the patient's cartilage or other body parts.

Furthermore, the present invention provides a prosthesis that is safe against bacterial infection and is rigid compared to the autologous cartilage, which is vulnerable to bacterial infection and is not sufficiently rigid, so that shape and function of the patient's nose may be maintained after surgery.

Hereinafter, various embodiments according to the present invention will be described in detail with reference to the accompanying drawings. The configuration of the present invention and effect thereof will be clearly understood through the following detailed description.

First Embodiment

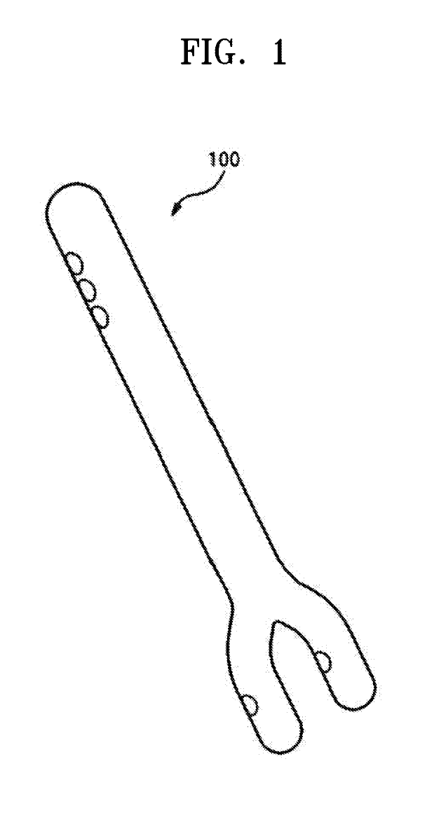

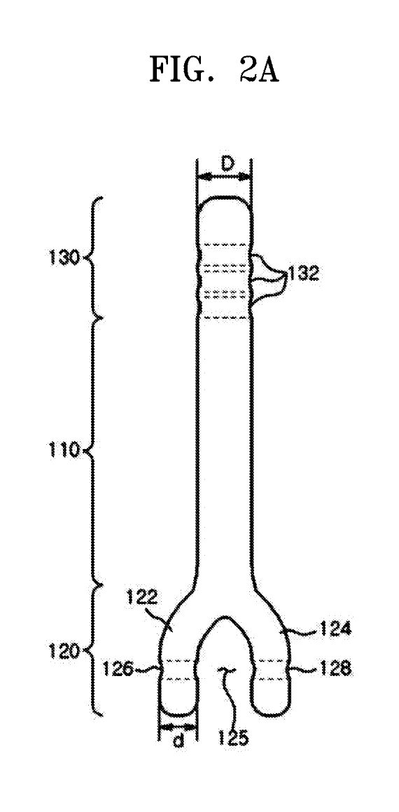

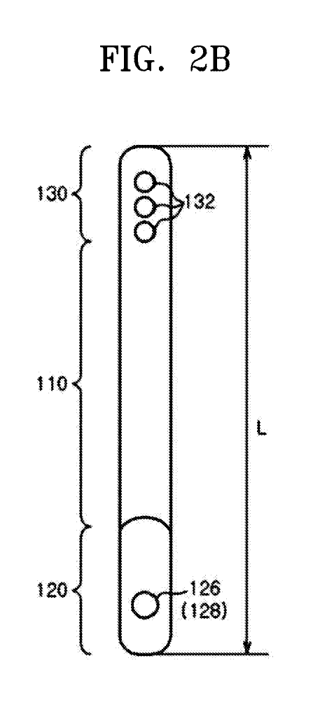

FIG. 1 is a perspective view of a prosthesis 100 for nose surgery according to the present invention, FIG. 2A is a plan view of a prosthesis 100 according to a first embodiment of the present invention, and FIG. 2B is a side view of the prosthesis 100 according to the first embodiment of the present invention.

Referring to FIGS. 1, 2A, and 2B, the prosthesis 100 according to the first embodiment of the present invention has a generally inverted ‘Y’ shape. The prosthesis 100 includes a column part 110 which is disposed in a columella 60 along a longitudinal direction of the nose when inserted into the nose, a first fixing part 120 which is connected to one end of the column part 110, and a second fixing part 130 which is connected to the other end of the column part 110. The second fixing part 130 is elongated from the column part 110, for example, in a cylindrical shape. A diameter of the column part 110 and the second fixing part 130 may be about 2 mm to 4 mm.

The column part 110 and the second fixing part 130 may be linear in a straight line, but are not limited thereto. The column part 110 and the second fixing part 130 may have a curved shape along an outer surface of the columella 60. A length of the prosthesis 100 of the present invention including the first fixing part 120, the column part 110, and the second fixing part 130 may be 1 cm to 5 cm.

As shown in FIG. 2A, the first fixing part 120 includes a first leg 122 and a second leg 124 which are divided from one end of the column part 110 to be extended in ‘Y’ shape, and a fixing groove 125 defined by a space between the first and second legs 122 and 124.

The first and second legs 122 and 124 may have cylindrical shape or a shape of a flat plate. As shown in FIG. 2A, in the first and second legs 122 and 124, a width d (or a diameter) of a cross section of the first or second legs 122 or 124 may be smaller than a width D (or diameter) of a cross section of the column part 110 or the second fixing part 130.

In addition, as shown in FIGS. 2A and 2B, in the first fixing part 120, a first through hole 126 passing through the first leg 122 is formed at the first leg 122, and a second through hole 126 passing through the second leg 124 is formed at the second leg 124. The first and second through holes 126 and 128 have the same penetrating direction, a central axis of the first through hole and a central axis of the second through holes are collinear, and therefore, as described below, a surgical needle and a suture may pass through the first and second through holes 126 and 128 at a time.

As shown in FIGS. 2A and 2B, the second fixing part 130 is connected to the other end of the column part 110, that is, an opposite end of the end to which the first fixing part 120 is connected. A third through hole 132 passing through the second fixing part 130 is formed in the second fixing part 130.

As illustrated in FIG. 2B, a plurality of third through holes 132 (e.g., three holes) may be formed along a longitudinal direction of the second fixing part 130 and the column part 110. Since a length of the columella 60 (a height of the nose) varies from patient to patient, a distance between a posterolateral part 210 of the nasal septal cartilage 200 and the alar cartilages 300 varies. As the plurality of third through holes 132 are formed in the second fixing part 130 along the longitudinal direction of the second fixing part 130, the most suitable third through hole 132 among the third through holes 132 is selected based on the length of the columella 60 and a position of the alar cartilages 300 for the patient, and then a suture may pass through the most suitable third through hole 132. Therefore, the second fixing part 130 may be properly coupled between the alar cartilages 300.

The suture passes through at least two of the third through holes 132 among the plurality of third through holes 132 to more firmly couple the second fixing part 130 to the alar cartilages 300. Further, when a plurality of third through holes 132 are formed, all of the plurality of third through holes 132 may be filled with a living tissue to be integrally adhered to a surrounding tissue as described later, so that the second fixing part 130 may be solidly coupled to the alar cartilages 300 compared to only one third through hole 132.

In the first embodiment of the present invention, the prosthesis 100 is made of a ceramic material. The ceramic material constituting the prosthesis 100 of the present invention is a material generally used for a prosthesis inserted into a human body such as a dental implant. An example of the ceramic material may include a zirconia material. As a further example of the ceramic material, VINTAGE HALO Porcelain powder manufactured by SHOFU Inc. of Japan, and its exemplary glass component is as follows.
SiO₂: 60 to 68%
Al2O3: 15 to 18%
K2O: 9 to 11%
Na2O: 7 to 8%
CaO: 0.5 to 2%
The rest: 0 to 5%

The ceramic, a high-strength material, may withstand force which intends to return to an original low nose state, that is, a downward pressure, after surgery and may be not deformed by the downward pressure. Also, since the prosthesis 100 is not easily deformed or damaged by external impact, the shape and function of the patient's nose is preserved without being damaged after surgery.

Hereinafter, a configuration in which the prosthesis 100 according to the present invention is inserted into the nose to be coupled and fixed to surrounding tissues will be described in detail.

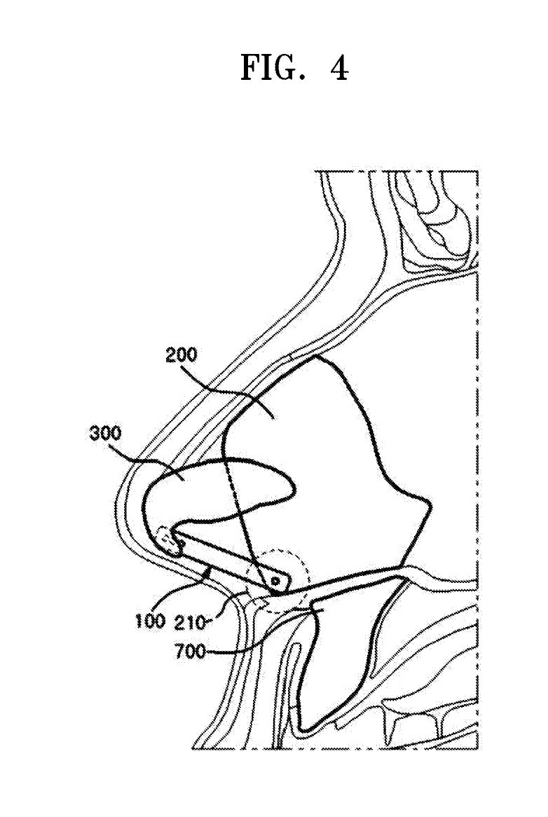

First, a configuration in which the first fixing part 120 is coupled to the posterolateral part 210 of the nasal septal cartilage 200 will be described. As shown in FIG. 2A, the first fixing part 120 includes the fixing groove 125 formed between the first leg 122 and the second leg 124. As shown in FIGS. 3 and 4, the posterolateral part 210 of the nasal septal cartilage 200 may be inserted into the fixing groove 125.

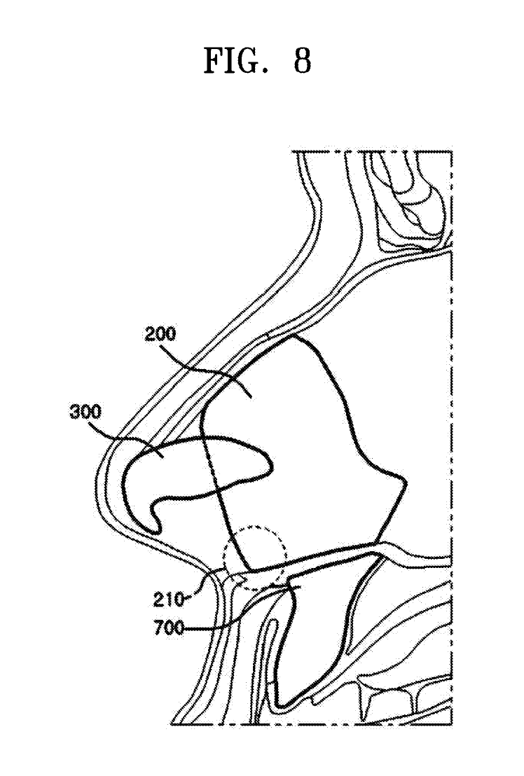

As shown in FIGS. 4 and 8, an anterior nasal spine 700 made of hard bone (tibia) is disposed below the nasal septal cartilage 200. The posterolateral part 210 of the nasal septal cartilage 200 is a part closest to the anterior nasal spine 700 in the nasal septal cartilage 200. The nasal septal cartilage 200 is a cartilage in the form of a wide plate with different thickness or strength depending on regions. The posterolateral part 210 is a thick and strong portion of the nasal septal cartilage 200 compared to other parts and is close to the anterior nasal spine 700 made of the tibia. When the first fixing part 120 is coupled to the posterolateral part 210 of the nasal septal cartilage 200, one end of the prosthesis 100 may be stably fixed within the nose.

Referring to FIG. 3, in the first fixing part 120, a suture made of a biodegradable polymer material passes through the first through hole 126, the posterolateral part 210 of the nasal septal cartilage 200, and the second through hole 128 in sequence or in reverse order, so that the first fixing part 120 may be sutured with the posterolateral part 210 of the nasal septal cartilage 200. Here, since the posterolateral part 210 of the nasal septal cartilage 200 is cartilage, a surgical needle and the suture may pass through the posterolateral part 210 of the nasal septal cartilage 200 without forming a separate through hole in the posterolateral part 210 of the nasal septal cartilage 200. As the first and second through holes 126 and 128 are already formed in the first and second legs 122 and 124, respectively, coupling the first fixing part 120 to the posterolateral part 210 of the nasal septal cartilage 200 using the suture may proceed with a simple procedure.

Furthermore, according to the configuration of the present invention, the first fixing part 120 and the posterolateral part 210 of the nasal septal cartilage 200 are overlapped and sutured together in a three-layered way, thereby being firmly coupled to one another.

The suture used in the present invention is harmless to the human body since the suture is inserted into the living body, and various sutures known in the art may be selected and used.

When a predetermined period of time, for example, about 6 months, elapses after surgery, the suture is decomposed and disappears in the body, the first and second through holes 126 and 128 are filled with a living tissue to be integrally adhered to a surrounding tissue, e.g., the posterolateral part 210 of the nasal septal cartilage 200. As a result, the first fixing part 120 may be stably adhered and fixed to the posterolateral part 210 of the nasal septal cartilage 200.

Subsequently, the second fixing part 130 is coupled between the both alar cartilages 300a and 300b. As shown in FIG. 3, the prosthesis 100, in which the first fixing part 120 is coupled to the posterolateral part 210 of the nasal septal cartilage 200 by the suture, extends along a longitudinal direction of the nose within the columella 60, so that the second fixing part 120 is disposed between the alar cartilages 300a and 300b. Referring to FIG. 3, in detail, the second fixing part 130 is disposed between both inner cartilages 320a and 320b of the alar cartilages 300a and 300b, respectively.

Here, in the second fixing part 130, a suture passes through one of the alar cartilage 300a, at least one of the third through holes 132, and the other alar cartilage 300b in order or in reverse order, and therefore, the second fixing part 130 may be fixed to the both alar cartilages 300a and 300b. Since the both alar cartilages 300a and 300b are cartilage, a surgical needle and the suture may pass through the both alar cartilages 300a and 300b without forming an additional through hole in the alar cartilages 300a and 300b. At least one of the third through holes 132 are formed in the second fixing part 130 already, so that coupling the second fixing part 130 to the alar cartilages 300a and 300b using the suture may proceed with a simple procedure.

Since the length of the columella 60 (a height of the nose) varies from patient to patient, the distance between the posterolateral part 210 of the nasal septal cartilage 200 and the alar cartilages 300 varies. In the present invention, as the plurality of third through holes 132 are formed in the second fixing part 130 along the longitudinal direction of the second fixing part 130, the most suitable third through hole 132 among the third through holes 132 is selected based on the length of the columella 60 and the position of the alar cartilages 300 for the patient, and then the suture may pass through the most suitable third through hole 132.

With this configuration, when the second fixing part 130 is coupled between the alar cartilages 300, it is possible that the suture may pass through an optimum position. Also, a plurality of third through holes 132 are formed along the longitudinal direction of the second fixing part 130 so that the suture passes through at least two of the third through holes 132 among the plurality of third through holes 132 to more firmly fix the second fixing part 130 to the alar cartilages 300. Further, when a plurality of third through holes 132 are formed, all of the plurality of third through holes 132 may be filled with a living tissue to be adhered to a surrounding tissue, so that the second fixing part 130 may be solidly fixed to the alar cartilages 300 compared to only one third through hole 132.

In addition, according to the configuration of the present invention, the second fixing part 130 and the alar cartilages 300 are overlapped and sutured together in a three-layered way, thereby being firmly coupled one another.

As described above, the suture used in the present invention is harmless to the human body since the suture is inserted into the living body, and various sutures known in the art may be selected and used. When a predetermined period of time, for example, about 6 months, elapses after surgery, the suture is decomposed and disappears in the body, the third through holes 132 are filled with the living tissue to be integrally adhered to the surrounding tissue, e.g., the alar cartilages 300a and 300b. As a result, the second fixing part 130 may be stably adhered and fixed to the both of the alar cartilages 300a and 300b.

As described above, by using the prosthesis 100 for nose surgery according to the present invention, it is not necessary to take the nasal septal cartilage 200 or the costal cartilage from the patient for reinforcing the columella 60, the autologous cartilage is reserved, and therefore malfunction or defunctionalization of the autologous cartilage is prevented.

Also, in the case of using the prosthesis 100 for nose surgery according to the present invention, since it is not necessary to take the costal cartilage which is an autologous cartilage, the procedure of nose surgery may be simplified. In addition, since it is not necessary to incise the skin around the costa besides the nose, nose surgery may be performed without scars around the patient's costa.

Second Embodiment

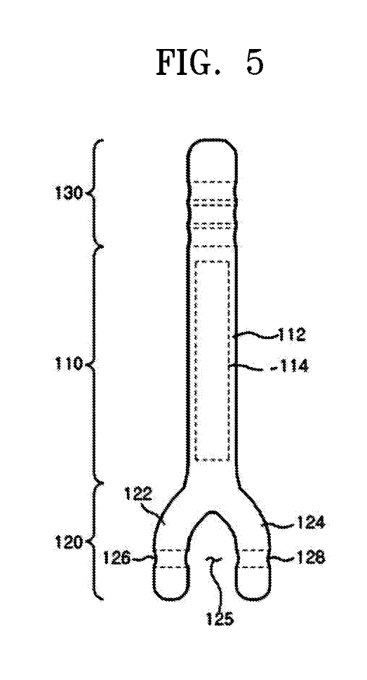

FIG. 5 shows a second embodiment to which the present invention is applied. The second embodiment of the present invention is the same as the configuration of the first embodiment except that a column part 110 includes an outer shell portion 112 and a core portion 114. Hereinafter, the configuration of the second embodiment which is different from the first embodiment will be described, and the remaining description thereof will be omitted.

Referring to FIG. 5, the column part 110 of the prosthesis 100 according to the second embodiment includes the outer shell portion 112 formed of a first material and the core portion 114 formed of a second material.

The second material forming the core portion 114 is a biomedical metal material commonly used in a prosthesis, which is inserted into a human body, such as a dental implant. The second material may include a titanium alloy, a cobalt alloy, stainless steel, and the like, which have high strength and excellent corrosion resistance. One specific example of a biomedical material is StarLoy N manufactured by DeguDent GmbH, Germany, and the composition on ingredients is as follows.
nickel: 61.0%
chromium: 25.8%
molybdenum: 11.0%
silicium: 1.5%
tungsten: 0 to 1.0%
aluminum: 0 to 1.0%

In addition, the first material forming the outer shell portion 112 is a ceramic material forming the prosthesis 100 in the first embodiment. For example, as described above, the first material may include VINTAGE HALO Porcelain powder manufactured by SHOFU Inc. of Japan.

The column part 110 of the second embodiment may be manufactured by a method in which the first material being the biomedical metal material is coated on the first material being the ceramic material. In detail, the column part 110 may be formed by a diffusion method, a spraying method, a chemical vapor deposition, and the like, which are known, but the present invention is not limited thereto.

In the second embodiment, the remaining configuration except for the core portion 114 of the column part 110, that is, the first fixing part 120 and the second fixing part 130 may be formed of the same ceramic material as the outer shell portion 112.

Since the core portion 114 of the column part 110 is made of the biomedical metal as described above, the prosthesis 100 according to the second embodiment of the present invention is further rigid, and therefore the prosthesis 100 may withstand the downward pressure, in which the nose returns to the original low nose state after surgery, and external impact. As a result, the shape and function of the patient's nose may be maintained more stably without any damage after surgery.

The terminology used herein is for the purpose of describing particular embodiments only and is not intended to be limiting of the invention. The singular expressions include plural expressions unless the context clearly dictates otherwise. In the present application, the terms “comprises” or “having” and the like are used to specify that there is a feature, a number, a step, an operation, an element, a component or a combination thereof described in the specification, but do not preclude the presence or addition of one or more other features, integers, steps, operations, elements, components, or combinations thereof.

While the present invention has been particularly shown and described with reference to exemplary embodiments thereof, it is to be understood that the invention is not limited to the disclosed exemplary embodiments. It will be understood by those skilled in the art that various changes in form and details may be made therein without departing from the spirit and scope of the present invention.

### INDUSTRIAL APPLICABILITY

The present invention is industrially applicable in industrial fields related to medical materials.

What is claimed is:

1. A prosthesis 100 for nose surgery comprising:
a column part 110;
a first fixing part 120 including a first leg 122 and a second leg 124 which are divided from one end of the column part 110 to be extended; and
a second fixing part 130 disposed at the other end of the column part 110.

2. The prosthesis of claim 1,
wherein the column part 110 is disposed in a columella 60 when being inserted into the nose.

3. The prosthesis of claim 1,
wherein the first fixing part 120 is fixed to a posterolateral part 210 of a nasal septal cartilage 200.

4. The prosthesis of claim 1,
wherein the second fixing part 130 is fixed between both alar cartilages 300.

5. The prosthesis of claim 3,
wherein the second fixing part 130 further includes a fixing groove 125 formed between the first and second legs 122 and 124 and the posterolateral part 210 of the nasal septal cartilage 200 is coupled to the fixing groove 125.

6. The prosthesis of claim 3,
wherein the first leg 122 has a first through hole 126 passing through the first leg 122, the second leg 124 has a second through hole 128 passing through the second leg 124, and a central axis of the first through hole 126 and a central axis of the second through hole 128 are collinear.

7. The prosthesis of claim 4,
wherein the second fixing part 130 has a third through hole 132 passing through the second fixing part 130.

8. The prosthesis of claim 7,
wherein a plurality of the third through holes 132 is formed in the column part 110 along a longitudinal direction of the second fixing part 130.

9. The prosthesis of claim 1,
wherein the column part 110 includes an outer shell portion 112 formed of a first material and a core portion 114 formed of a second material, and the outer shell portion 112 surrounds a surface of the core portion 114.

10. The prosthesis of claim 9,
wherein the second material includes a biomedical metal.

11. The prosthesis of claim 6,
wherein the first fixing part 120 is fixed to the posterolateral part 210 of the nasal septal cartilage 200 by a suture passing through the first through hole 126, the posterolateral part 210 of the nasal septal cartilage 200, and the second through hole 128.

12. The prosthesis of claim 11,
wherein the suture is decomposed and disappears in a body when a predetermined period of time elapses, and the first and second through holes 126 and 128 are filled with a living tissue to be integrally adhered to a surround tissue.

13. The prosthesis of claim 8,
wherein the second fixing part 130 is fixed between both alar cartilages 300 by a suture passing through one alar cartilage 300a, at least one of the third through holes 132, and the other alar cartilage 300b.

14. The prosthesis of claim 13,
wherein the suture is decomposed and disappears when a predetermined period of time elapses, and the third through holes 132 are filled with a living tissue to be integrally adhered to a surround tissue.
